(19) United States
(12) Patent Application Publication
(10) Pub. No.: US 2023/0329873 A1
(43) Pub. Date: Oct. 19, 2023

(54) HIP JOINT DEVICE AND METHOD

(71) Applicant: Peter Forsell, Bouveret (CH)

(72) Inventor: Peter Forsell, Bouveret (CH)

(21) Appl. No.: 18/214,270

(22) Filed: Jun. 26, 2023

Related U.S. Application Data
(63) Continuation of application No. 13/383,274, filed on Jan. 10, 2012.
(60) Provisional application No. 61/229,739, filed on Jul. 30, 2009.
(60) Provisional application No. 61/229,743, filed on Jul. 30, 2009.
(60) Provisional application No. 61/229,745, filed on Jul. 30, 2009.
(60) Provisional application No. 61/229,746, filed on Jul. 30, 2009.
(60) Provisional application No. 61/229,747, filed on Jul. 30, 2009.
(60) Provisional application No. 61/229,748, filed on Jul. 30, 2009.
(60) Provisional application No. 61/229,751, filed on Jul. 30, 2009.
(60) Provisional application No. 61/229,752, filed on Jul. 30, 2009.
(60) Provisional application No. 61/229,755, filed on Jul. 30, 2009.
(60) Provisional application No. 61/229,761, filed on Jul. 30, 2009.
(60) Provisional application No. 61/229,767, filed on Jul. 30, 2009.
(60) Provisional application No. 61/229,778, filed on Jul. 30, 2009.
(60) Provisional application No. 61/229,786, filed on Jul. 30, 2009.
(60) Provisional application No. 61/229,789, filed on Jul. 30, 2009.
(60) Provisional application No. 61/229,796, filed on Jul. 30, 2009.
(60) Provisional application No. 61/229,735, filed on Jul. 30, 2009.

(30) Foreign Application Priority Data
Apr. 10, 2009 (SE) 0900968-9
Jul. 10, 2009 (SE) 0900693-0
Jul. 10, 2009 (SE) 0900957-2
Jul. 10, 2009 (SE) 0900958-0
Jul. 10, 2009 (SE) 0900959-8
Jul. 10, 2009 (SE) 0900960-6
Jul. 10, 2009 (SE) 0900962-2
Jul. 10, 2009 (SE) 0900965-5
Jul. 10, 2009 (SE) 0900966-3
Jul. 10, 2009 (SE) 0900969-7
Jul. 10, 2009 (SE) 0900970-5
Jul. 10, 2009 (SE) 0900972-1
Jul. 10, 2009 (SE) 0900973-9
Jul. 10, 2009 (SE) 0900974-7
Jul. 10, 2009 (SE) 0900978-8
Jul. 10, 2009 (SE) 0900981-2
Jul. 10, 2009 (SR) 0900976-2

(51) Int. Cl. A61F 2/32; A61B 17/16; A61F 2/36

(52) U.S. Cl. CPC A61F 2/32; A61B 17/1668; A61B 17/1666; A61F 2/3603; A61F 2002/30818

(57) ABSTRACT

A method of treating a hip joint of a human patient by providing said the medical device is provided. The hip joint comprising a caput femur and an acetabulum, the method comprises the steps of: cutting the skin of the patient, dissecting an area of the pelvic bone on the opposite side from the acetabulum, creating a hole in said dissected area, said hole passing through said pelvic bone and into the hip joint of the patient, and providing said medical device to the hip joint, through said hole in the pelvic bone of the patient.

### FIELD OF INVENTION

The present invention relates generally to a medical device for implantation in a hip joint, and a method of providing said medical device.

### BACKGROUND

Hip joint Osteoarthritis is a syndrome in which low-grade inflammation results in pain in the hip joints, caused by abnormal wearing of the Cartilage that acts as a cushion inside if the hip joint. This abnormal wearing of the cartilage also results in a decrease of the joints lubricating fluid called Synovial fluid. Hip joint Osteoarthritis is estimated to affect 80% of all people over 65 years of age, in more or less serious forms.

The present treatment for hip osteoarthritis comprises NSAID drugs, local injections of Hyaluronic acid or Glucocorticoid to help lubricating the hip joint, and replacing parts of the hip joint with a prosthesis through hip joint surgery.

The replacing of parts of the hip joint is one of the most common surgeries to date performed at hundreds of thousands of patients in the world every year. The most common method comprises placing a metal prosthesis in Femur and a plastic bowl in Acetabulum. This operation is usually done through a lateral incision in the hip and upper thigh and through, Fascia Lata and the lateral muscles of the thigh. To get access to the hip joint, the supporting hip joint capsule attached to Femur and Ilium of Pelvis needs to be penetrated, making it difficult to get a fully functional joint after the surgery. Femur is then cut at the neck with a bone saw and the prosthesis is placed in femur either with bone cement or without. Acetabulum is slightly enlarged using an Acetabular reamer, and the plastic bowl is positioned using screws or bone cement.

The surgery typically requires one week of hospitalization due to the increased risk of infection. The recovery process is on average about 6 weeks, but even after this period the patient should not perform any physical activates that places large strain on the joint.

### SUMMARY

A medical device for treating hip joint osteoarthritis in a human patient by providing at least one artificial hip joint surface is provided. The hip joint having a ball shaped caput femur as the proximal part of the femoral bone with a convex hip joint surface towards the centre of the hip joint and a bowl shaped acetabulum as part of the pelvic bone with a concave hip joint surface towards the centre of the hip joint. The medical device comprising the artificial hip joint surface comprises at least one of; an artificial caput femur or an artificial caput femur surface comprising, a convex form towards the centre of the hip joint, and an artificial acetabulum or an artificial acetabulum surface comprising, a concave form towards the centre of the hip joint. The artificial convex caput femur or the artificial convex caput femur surface is adapted to be fixated to the pelvic bone of the human patient, and the artificial concave acetabulum or artificial concave acetabulum surface is adapted to be fixated to the femoral bone of the human patient.

Alternatively a medical device for treating hip joint osteoarthritis by providing at least one joint surface is provided. The hip joint has a ball shaped caput femur as the proximal part of the femoral bone with a convex hip joint surface and a bowl shaped acetabulum as part of the pelvic bone with a concave hip joint surface.

The medical device could comprise an artificial convex hip joint surface adapted to replace the convex hip joint surface. The artificial convex hip joint surface could be adapted to be fixated to the pelvic bone of the human patient.

According to one embodiment, the medical device comprises an artificial convex hip joint surface adapted to be inserted through a hole in the pelvic bone of the human patient.

According to one embodiment, the medical device comprises an artificial convex hip joint surface adapted to be inserted through a hole in the hip joint capsule of the human patient.

According to one embodiment, the medical device comprises an artificial convex hip joint surface adapted to be inserted through a hole in the femoral bone of the human patient.

The medical device could be adapted to be placed in an artificial replacement of the concave acetabulum hip joint surface. The artificial replacement could be adapted to be fixated to the caput femur, the collum femur or the femoral bone.

According to one embodiment the artificial convex hip joint surface in the medical device comprises at least two artificial hip joint surface parts adapted to be placed in connection with each other after the insertion in the human patient. The at least two artificial hip joint surface parts could be adapted to be inserted through a hole in the pelvic bone from the opposite side from acetabulum of the human patient, said hole having a diameter less than the largest diameter of said medical device. It is also conceivable that the at least two artificial hip joint surface parts are adapted to be inserted through a hole in the femoral bone or the hip joint capsule of the human patient. The hole having a diameter smaller than the largest diameter of the medical device.

According to any of the embodiments above, at least one of said at least two artificial hip joint surface parts could be adapted to serve as base part to which at least one additional artificial caput femur surface part can be connected.

The connection of said at least two artificial hip joint surface parts could be performed using at least one of: at least one screw, at least one pin, at least one portion of at least one of the parts adapted to be introduced into the other part, the parts being adapted to be sliding into the other part, form fitting, welding, adhesive, pin, wire, a ball mounted into a bowl being portions of said parts, a male portion of one part mounted into a female portion of the other part, a key introduced into a lock being portions of said parts, band, or other mechanical connecting members. It is also conceivable that the at least two artificial hip joint surface parts are adapted to mechanically connect to each other using self locking elements, in which case the self locking elements could be assisted by adhesive or bone cement.

According to one embodiment, the medical device comprises at least one elastic member. The at least one elastic member could be adapted for changing the largest diameter or largest cross-sectional distance of the medical device for insertion through a hole having a diameter smaller than said largest diameter or cross-sectional distance of said medical device.

### Fixation

To fixate the medical device it is conceivable that the medical device comprises a fixation support to anchor the artificial convex hip joint surface. The fixation support is adapted to anchor the artificial convex hip joint surface to the pelvic bone, to support at least part of the load applied to the hip joint in normal use.

According to one embodiment the fixation support comprises a displaceable part or section. The displaceable part or section could be adapted to carry the load applied to the hip joint in normal use.

According to one embodiment the medical device could be adapted to be fixated to the pelvic bone using at least one of: at least one screw, at least one pin, at least one portion of at least one of the parts adapted to be introduced into the other part, the parts being adapted to be sliding into the other part, form fitting, welding, adhesive, pin, wire, a ball mounted into a bowl being portions of said parts, a male portion of one part mounted into a female portion of the other part, a key introduced into a lock being portions of said parts, band, or other mechanical connecting members.

According to one embodiment the medical device could be fixated to the pelvic bone without penetration of the cortex of the pelvic bone. It is also conceivable that the medical device is adapted to be fixated to the pelvic bone by means of said elastic member exerting a clamping force on the pelvic bone.

### Material

The medical device according to any of the embodiments above could comprise at least one of the materials: polyethylene based material, PTFE, Corian, titanium, stainless steel, wolfram, other metal material, a combination of metal material, carbon fiber, boron, a combination of metal and plastic materials, a combination of metal and carbon based material, a combination of carbon and plastic based material, multi-material, wherein one material comprise a flexible material, multi-material, wherein one material comprise an elastic material, multi-material, wherein one material comprising more parts than the other at least one material, PE, and an acrylic polymer. It is also conceivable that the medical device comprises a self lubricating material. In cases where the medical device do not comprise a self lubricating material or if the self lubricating material is not sufficient it is conceivable that the medical device is adapted to be lubricated after insertion in the hip joint.

According to one embodiment the medical device could be adapted to be lubricated after insertion in the hip joint. It is furthermore conceivable that the medical device comprises a self lubricating material such as PTFE.

### The Acetabulum Surface

The present invention further relates to a medical device for treating hip joint osteoarthritis in a human patient by providing at least one artificial hip joint surface. The hip joint has a ball shaped caput femur as the proximal part of the femoral bone with a convex hip joint surface and a bowl shaped acetabulum as part of the pelvic bone with a concave hip joint surface.

The medical device comprises an artificial concave hip joint surface, adapted to be fixated to at least one of: caput femur, collum femur or another part of the femoral bone of the human patient.

According to one embodiment, the medical device comprises an artificial concave hip joint surface adapted to be inserted through a hole in the pelvic bone of the human patient.

According to one embodiment, the medical device comprises an artificial concave hip joint surface adapted to be inserted through a hole in the hip joint capsule of the human patient.

According to one embodiment, the medical device comprises an artificial concave hip joint surface adapted to be inserted through a hole in the femoral bone of the human patient. According to one embodiment the artificial concave hip joint surface is further adapted to be placed in connection with an artificial replacement of the convex hip joint surface. The artificial replacement could be adapted to be fixated to the pelvic bone.

According to one embodiment the artificial concave hip joint surface comprises at least two artificial hip joint surface parts adapted to be placed in connection with each other after the insertion in the human patient.

The at least two artificial hip joint surface parts could be adapted to be inserted through a hole in the pelvic bone from the opposite side from acetabulum of the human patient, said hole having a diameter less than the largest diameter of said medical device. It is also conceivable that the at least two artificial hip joint surface parts are adapted to be inserted through a hole in the femoral bone or the hip joint capsule of the human patient. The hole having a diameter less than the largest diameter of said medical device.

According to any of the embodiments above, at least one of said at least two artificial hip joint surface parts could be adapted to serve as base part to which at least one additional artificial caput femur surface part can be connected.

The connection of said at least two artificial hip joint surface parts could be performed using at least one of: at least one screw, at least one pin, at least one portion of at least one of the parts adapted to be introduced into the other part, the parts being adapted to be sliding into the other part, form fitting, welding, adhesive, pin, wire, a ball mounted into a bowl being portions of said parts, a male portion of one part mounted into a female portion of the other part, a key introduced into a lock being portions of said parts, band, or other mechanical connecting members. It is also conceivable that the at least two artificial hip joint surface parts are adapted to mechanically connect to each other using self locking elements, in which case the self locking elements could be assisted by adhesive or bone cement.

According to one embodiment of the present invention the medical device comprises at least one elastic member. The at least one elastic member could be adapted for changing the largest diameter or largest cross-sectional distance of the medical device for insertion through a hole having a diameter smaller than said largest diameter or cross-sectional distance of said medical device.

### Fixation

To fixate the medical device according to the present invention it is conceivable that the medical device comprises a fixation support to anchor said artificial concave hip joint surface. The fixation support is adapted to anchor said artificial concave hip joint surface to the caput femur, the collum femur or another part of the femoral bone of the human patient, to at least partly support the load applied to the hip joint in normal use.

The medical device according to claim 40, wherein said fixation support comprises a displaceable part or section, and wherein said displaceable part or section is adapted to carry the load applied to the hip joint in normal use.

According to one embodiment the medical device could be adapted to be fixated to caput femur, the collum femur or the femoral bone using at least one of: at least one screw, at least one pin, at least one portion of at least one of the parts adapted to be introduced into the other part, the parts being adapted to be sliding into the other part, form fitting, welding, adhesive, pin, wire, a ball mounted into a bowl being portions of said parts, a male portion of one part mounted into a female portion of the other part, a key introduced into a lock being portions of said parts, band, or other mechanical connecting members. It is also conceivable that the fixation is done using self locking elements, in which case the self locking elements could be assisted by adhesive or bone cement.

According to one embodiment the medical device could be fixated to the caput femur, the collum femur or the femoral bone without penetration of the cortex of the pelvic bone. It is also conceivable that the medical device is adapted to be fixated to the caput femur, the collum femur or the femoral bone by means of said elastic member exerting a clamping force on the caput femur, collum femur or femoral bone.

### Material

The medical device according to any of the embodiments could comprise at least one of the materials: polyethylene based material, PTFE, Corian, titanium, stainless steel, wolfram, other metal material, a combination of metal material, carbon fiber, boron, a combination of metal and plastic materials, a combination of metal and carbon based material, a combination of carbon and plastic based material, multi-material, wherein one material comprise a flexible material, multi-material, wherein one material comprise an elastic material, multi-material, wherein one material comprising more parts than the other at least one material, PE, and an acrylic polymer. It is also conceivable that the medical device comprises a self lubricating material. In cases where the medical device do not comprise a self lubricating material or if the self lubricating material is not sufficient it is conceivable that the medical device is adapted to be lubricated after insertion in the hip joint.

### The System

The present invention further relates to a medical device system for treating hip joint osteoarthritis in a human patient by providing at least two hip joint surfaces. The system comprising the artificial convex hip joint surface according to any of the embodiments above and the artificial concave hip joint surface according to any of the embodiments above.

The artificial convex hip joint surface in the medical device system could be adapted to be placed, at least partly, inside of said artificial concave hip joint surface.

According to one embodiment the artificial convex hip joint surface comprises a largest diameter and said artificial concave hip joint surface could be adapted to travel beyond the largest diameter of the artificial convex hip joint surface.

According to one embodiment the medical device system has a ball shaped caput femur as the proximal part of the femoral bone with a convex hip joint surface, and a bowl shaped acetabulum as part of the pelvic bone with a concave hip joint surface. The artificial convex hip joint surface could be adapted to be placed, at least partly, inside of the caput femur.

According to one embodiment the medical device could be adapted to be lubricated after insertion in the hip joint. Said lubrication is preferably done with a biocompatible lubricator such as hyaluronic acid. It is furthermore conceivable that the medical device comprises a self lubricating material such as PTFE.

### Centering Device

According to one embodiment the medical device comprising an artificial acetabulum or an artificial acetabulum surface, wherein said elongated member is adapted to centre and hold said artificial acetabulum or an artificial acetabulum surface during fixation in the hip joint.

According to one embodiment the medical device comprising an artificial caput femur or an artificial caput femur surface, wherein said elongated member is adapted to centre and hold said artificial caput femur or an artificial caput femur during fixation in the hip joint.

According to one embodiment said elongated member is adapted to centre and hold both said artificial caput femur or an artificial caput femur and said artificial acetabulum or an artificial acetabulum surface during fixation in the hip joint.

According to one further embodiment of the medical device, the artificial caput femur surface comprises a convex form towards the centre of the hip joint and the artificial acetabulum surface comprises a concave form towards the centre of the hip joint. The artificial caput femur surface and the artificial acetabulum surface is constructed to be placed in the hip joint in a opposite position towards each other, thus; the artificial convex caput femur surface is adapted to be fixated to the pelvic bone of the human patient, and the artificial concave acetabulum surface is adapted to be fixated to the femoral bone of the human patient.

According to one embodiment the artificial acetabulum or artificial acetabulum surface is adapted to be centered and held by said elongated member, during fixation in the hip joint.

According to one embodiment the artificial caput femur or an artificial caput femur surface is adapted to be centered and held by the elongated member, during fixation in the hip joint.

### The Method

A further aspect of the present invention is a method of treating hip joint osteoarthritis in a human patient by providing at least one hip joint surface. The method comprises the steps of: fixating the artificial convex hip joint surface, according to any of the embodiments above, to the pelvic bone, and fixating the artificial concave hip joint surface according to any of the embodiments above to at least one of; the caput femur, the collum femur or the femoral bone of the human patient.

The method could further comprise, the steps of: cutting the skin of the human patient, dissecting an area of the pelvic bone on the opposite side from the acetabulum, creating a hole in said dissected area, said hole passing through the pelvic bone and into the hip joint of the human patient, inserting said artificial hip joint surfaces into the hip joint through said hole. After the steps of fixating the artificial convex hip joint surface, according to any of the embodiments above, to the pelvic bone, and fixating the artificial concave hip joint surface according to any of the embodiments above to at least one of; the caput femur, the collum femur or the femoral bone of the human patient, the method further comprises the steps of: Closing the hole in the pelvic bone using bone material or a prosthetic part and closing, preferable in layers, the hip area of the human patient using sutures or staples.

According to a second embodiment the method is a method of treating hip joint osteoarthritis by providing artificial hip joint surfaces according to any of the embodiments above. The hip joint comprises a ball shaped caput femur as the proximal part of the femoral bone with a convex hip joint surface and a bowl shaped acetabulum as part of the pelvic bone with a concave hip joint surface, the method comprises the steps of: cutting the skin of the human patient, dissecting an area of the hip joint, creating a hole in the hip joint capsule, inserting said artificial hip joint surfaces into the hip joint through said hole in the hip joint capsule, fixating the artificial convex hip joint surface device to the pelvic bone, fixating the concave artificial hip joint surface to at least one of; the caput femur, the collum femur or the femoral bone, and closing, preferable in layers, the hip area of the human patient using sutures or staples or adhesive.

According to a third embodiment the method of treating hip joint osteoarthritis by providing artificial hip joint surfaces according to any of the embodiments above is a arthroscopic method. The hip joint comprises a ball shaped caput femur as the proximal part of the femoral bone with a convex hip joint surface and a bowl shaped acetabulum as part of the pelvic bone with a concave hip joint surface, the method comprises the steps of: inserting a needle or a tube like instrument into the patient's body, using the needle or tube like instrument to fill a part of the patient's body with gas and thereby expanding a cavity within said body, placing at least two arthroscopic trocars in said cavity, inserting a camera through one of the arthroscopic trocars into said cavity, inserting at least one dissecting tool through one of said at least two arthroscopic trocars, dissecting an area of the pelvic bone on the opposite side from the acetabulum, creating a hole in said dissected area, said hole passing through the pelvic bone and into the hip joint of the human patient, and providing said artificial hip joint surfaces to the hip joint, through said hole in the pelvic bone of the human patient.

According to a third embodiment the method of treating hip joint osteoarthritis by providing artificial hip joint surfaces according to any of the embodiments above is a second arthroscopic method. The hip joint comprises a ball shaped caput femur as the proximal part of the femoral bone with a convex hip joint surface and a bowl shaped acetabulum as part of the pelvic bone with a concave hip joint surface, the method comprises the steps of: inserting a needle or a tube like instrument into the patient's body, using the needle or tube like instrument to fill a part of the patient's body with gas and thereby expanding a cavity within said body, placing at least two arthroscopic trocars in said cavity, inserting a camera through one of the arthroscopic trocars into said cavity, inserting at least one dissecting tool through one of said at least two arthroscopic trocars, dissecting an area of the hip joint, creating a hole in the hip joint capsule, inserting said artificial hip joint surfaces into the hip joint through said hole in the hip joint capsule, fixating the artificial convex hip joint surface device to the pelvic bone, fixating the concave artificial hip joint surface to at least one of; the caput femur, the collum femur or the femoral bone, and closing, preferable in layers, the hip area of the human patient using sutures or staples or adhesive.

According to one embodiment the method of manipulation comprises the steps of; fixating an artificial acetabulum surface to the pelvic bone, wherein said elongated member; centers the artificial acetabulum surface, when the artificial acetabulum surface is fixated in the hip joint.

According to one embodiment the method of manipulation comprises the step of; fixating an artificial caput femur surface to the femoral bone, wherein said elongated member; centers said artificial caput femur surface, when said artificial caput femur surface is fixated in the hip joint.

According to one embodiment, a method of centering an artificial hip joint surface in a hip joint of a human patient is provided. The hip joint comprising a collum femur, being the proximal part of the femoral bone, a caput femur, being the upper extremity of the femoral bone, and an acetabulum, being a bowl shaped part of the pelvic bone, the method comprising the steps of: penetrating the skin of a lateral section of the thigh, creating a hole in the collum femur, along a length axis thereof, reaching an area of the hip joint, placing an elongated member in said hole, wherein said elongated member reaches centrally in said area of the hip joint, and centering said artificial hip joint surface onto said elongated member, wherein the artificial hip joint surface comprises a centre hole for guiding the elongated member, and placing the said artificial hip joint surface in a functional position in the hip joint.

The artificial hip joint surface, according to any of the embodiments above may comprise an artificial convex caput femur or an artificial convex caput femur surface or an artificial concave acetabulum or an artificial concave acetabulum surface.

Please note that any embodiment or part of embodiment as well as any method or part of method could be combined in any way. All examples herein should be seen as part of the general description and therefore possible to combine in any way in general terms.

### BRIEF DESCRIPTION OF DRAWINGS

The invention is now described, by way of example, with reference to the accompanying drawings, in which:

FIG. 1a shows the hip joint in section,

FIG. 1b shows the collum femur in section,

FIG. 2 shows the exposing of the caput femur through an incision in the thigh,

FIG. 3 shows the step of removing a proximal part of the caput femur,

FIG. 4 shows the reaming of the collum and caput femur,

FIG. 5 shows the collum and caput femur when a medical device gas been fixated,

FIG. 6 shows the reaming of the acetabulum,

FIG. 7 shows the injecting of an adhesive in the acetabulum,

FIG. 8 shows the fixation of a medical device in the acetabulum,

FIG. 9 shows an artificial hip joint, when connected,

FIG. 10 shows a medical device when anchored in the femoral bone,

FIG. 11 shows a frontal view of a human patient when incisions have been made in a surgical method,

FIG. 12 shows a frontal view of a human patient when incisions have been made in a arthroscopic method,

FIG. 13 shows the human patient in section when a medical device for creating a hole in the pelvic bone is inserted,

FIG. 14 shows the hip joint in section when a medical device for creating a hole in the pelvic bone is operating,

FIG. 15 shows the step of removing a proximal part of the caput femur,

FIG. 16 shows the hip joint in section when a reamer is introduced to a hole in the pelvic bone,

FIG. 17 shows the hip joint in section when an injecting member injects a fluid through a hole in the pelvic bone.

FIG. 18 shows the step of providing a medical device through a hole in the pelvic bone,

FIG. 19 shows the medical device in further detail,

FIG. 20 shows the hip joint in section when a medical device has been provided,

FIG. 21 shows the hip joint in section when a prosthetic part is being provided,

FIG. 22 shows the hip joint in section when the medical device has been fixated,

FIG. 23 shows a second approach to reaming the caput femur,

FIG. 24 shows the hip joint in section when an injecting member injects a fluid through a hole in the pelvic bone.

FIG. 25 shows a second approach to placing the medical device in the hip joint,

FIG. 26 shows a second approach to placing the medical device in the hip joint,

FIG. 27 shows the hip joint in section when a prosthetic part is being provided in a second approach,

FIG. 28 shows the hip joint in section when the medical device has been fixated,

FIG. 29 shows a schematic view of the concave hip joint surface in section,

FIG. 30 shows an artificial concave acetabulum surface in section,

FIG. 31 shows the step of injecting a fluid into an area of the hip joint or its surroundings.

### DETAILED DESCRIPTION

In the following a detailed description of preferred embodiments of the present invention will be given. In the drawing figures, like reference numerals designate identical or corresponding elements throughout the several figures. It will be appreciated that these figures are for illustration only and are not in any way restricting the scope of the invention. Thus, any references to direction, such as “up” or “down”, are only referring to the directions shown in the figures. Also, any dimensions etc. shown in the figures are for illustration purposes.

Functional hip movements are to be understood as movements of the hip that at least partly correspond to the natural movements of the hip. On some occasions the natural movements of the hip joint might be somewhat limited or altered after hip joint surgery, which makes the functional hip movements of a hip joint with artificial surfaces somewhat different than the functional hip movements of a natural hip joint.

The functional position of an implantable medical device or prosthesis is the position in which the hip joint can perform functional hip movements. The final position is to be understood as a functional position in which the medical device needs no further position change.

Arthroscopy is to be understood as key hole surgery performed in a joint, since the arthroscopic procedure could be performed in the abdomen of the patient some of the steps of this arthroscopic procedure is more laparoscopic, however for the purpose of this invention the two terms arthroscopy and laparoscopy is used synonymously and for the purpose of this invention the main purpose of these methods are is that they are minimally invasive.

The medical device according to any of the embodiments could comprise at least one material selected from a group consisting of: polytetrafluoroethylene (PTFE), perfluoroalkoxy (PFA) and fluorinated ethylene propylene (FEP). It is furthermore conceivable that the material comprises a metal alloy, such as cobalt-chromium-molybdenum or titanium or stainless steel, or polyethylene, such as cross-linked polyethylene or gas sterilized polyethylene. The use of ceramic material is also conceivable, in the contacting surfaces or the entire medical device such as zirconium or zirconium dioxide ceramics or alumina ceramics. The part of the medical device in contact with human bone for fixation of the medical device to human bone could comprise a poorhouse structure which could be a porous micro or nano-structure adapted to promote the growth-in of human bone in the medical device for fixating the medical device. The porous structure could be achieved by applying a hydroxy-apatite (HA) coating, or a rough open-pored titanium coating, which could be produced by air plasma spraying, a combination comprising a rough open-pored titanium coating and a HA top layer is also conceivable. The contacting parts could be made of a self lubricated material such as a waxy polymer, such as PTFE, PFA, FEP, PE and UHMWPE, or a powder metallurgy material which could be infused with a lubricant, which preferably is a biocompatible lubricant such as a Hyaluronic acid derivate. It is also conceivable that the material of contacting parts or surfaces of the medical device herein is adapted to be constantly or intermittently lubricated. According to some embodiments the parts or portions of the medical device could comprise a combination of metal materials and/or carbon fibers and/or boron, a combination of metal and plastic materials, a combination of metal and carbon based material, a combination of carbon and plastic based material, a combination of flexible and stiff materials, a combination of elastic and less elastic materials, Corian or acrylic polymers.

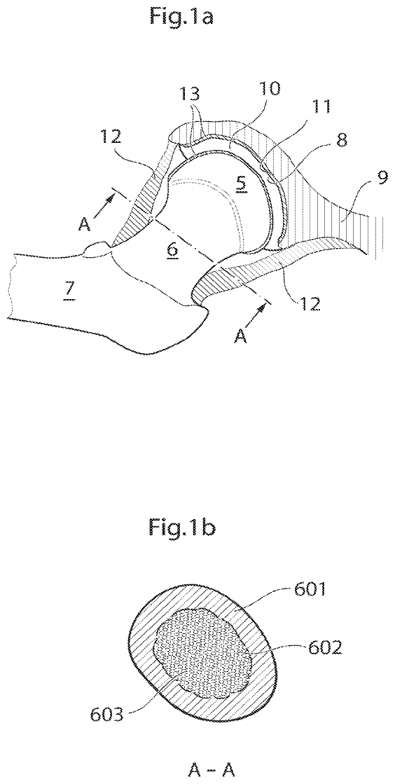

FIG. 1 shows the hip joint of a human patient in section. The hip joint comprises a caput femur 5 placed at the very top of collum femur 6 which is the top part of the femoral bone 7. The caput femur is in connection with the acetabulum 8, which is a bowl shaped part of the pelvic bone 9. Both the caput femur surface 10 and the acetabulum surface 11 is covered with articular cartilage 13 which acts as a cushion in the hip joint. In patients with hip joint osteoarthritis, this articular cartilage 13 is abnormally worn down due to a low grade inflammation. The hip joint is surrounded by the hip joint capsule 12 which provides support for the joint and hinders luxation. After conventional hip joint surgery, penetrating the hip joint capsule 12, the capsule 12 is dramatically weakened due to the limited healing possibilities of its ligament tissue. By performing hip joint surgery without damaging the hip joint capsule 12 the patient can fully recover and place equal amount of strain on an artificial joint as is possible on a natural one.

FIG. 1b shows a section A-A of the collum femur, as shown in FIG. 1. The section A-A shows the collum femur comprising cortical bone 601, the outer more sclerotic bone, and cancellous bone 602, the inner porous bone located in the bone marrow 603.

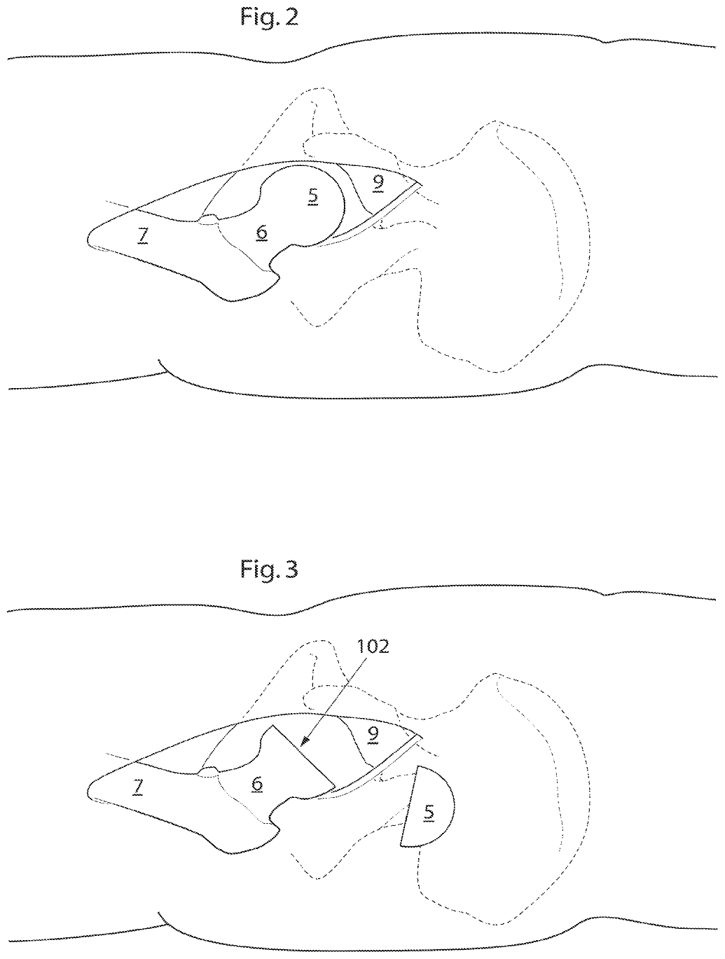

FIG. 2 shows a lateral view of a human patient when an incision in the thigh region has been made. The femoral bone 7 comprising the collum femur 6 and the caput femur 5 has been dislocated from its usual position in the hip joint, in connection with the acetabulum, which is a part of the pelvic bone 9, the caput femur 5 being a part of the hip joint normally being covered by the hip joint capsule.

FIG. 3 shows the proximal part of the caput femur 5 being removed e.g. by means of a bone saw. A surface of a section 102 is thus created perpendicularly to a length axis of the collum femur 6

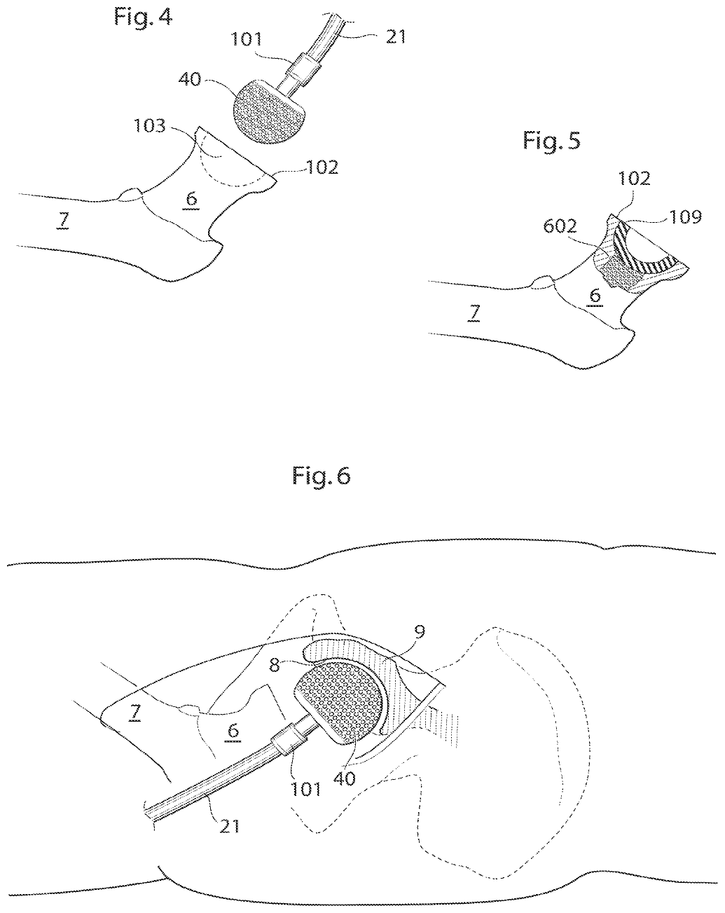

FIG. 4 shows the reaming of the collum femur 6 and caput femur 5 using a reamer 40 connecting to an elongated member 21 by a connecting section 101. The reamer 40 creating a hemi-spherical cavity, having a concave surface 103, centrally placed in the caput 5 and collum femur 6.

FIG. 5 shows the femoral bone 7 when a medical device 109 having a concave contacting surface has been provided to the hemi-spherical cavity, centrally placed in the caput 5 and collum femur.

FIG. 6 shows the reaming of the acetabulum 8 of the pelvic bone 9 using a reamer 40 connected to an elongated member 21 by means of a connecting section 101. The elongated member could be adapted to be flexible or bent to enable the reaching of the acetabulum 8. The reaming is needed to create enough space in the hip joint for the placing of a medical device, and for the preparing of the surfaces of the acetabulum 8 for the fixation of the medical device.

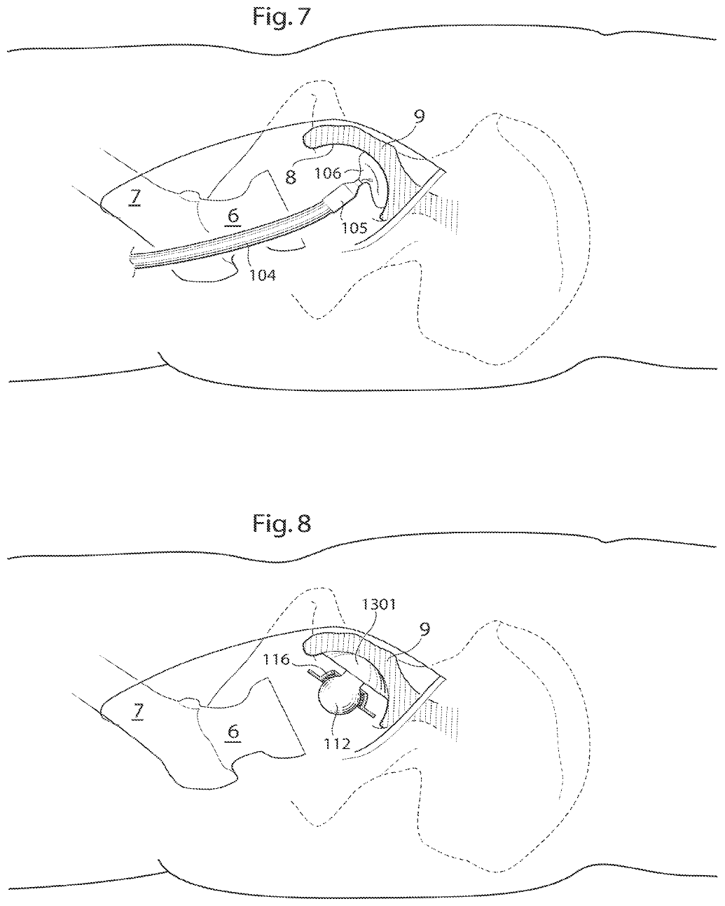

FIG. 7 shows the injection of a fluid 106, such as an adhesive or bone cement, into the reamed acetabulum 8 surface. The fluid 106 is injected using a injecting member 104 comprising an injecting nozzle 105.

FIG. 8 shows the placing of a medical device in the reamed acetabulum 8 surface of the pelvic bone 9. The medical device comprises a convex hip joint surface 112 fixated to a fixation part 1301, which in turn is fixated to the acetabulum 8 using the injected fluid, which could be assisted or replaced by a mechanical fixation element such as screws. The medical device further comprises a pre-mounted locking element 116 for locking the convex hip joint surface of the concave hip joint surface placed in the caput 5 and collum femur 6 for hindering dislocation of the hip joint when the hip joint is in its functional position.

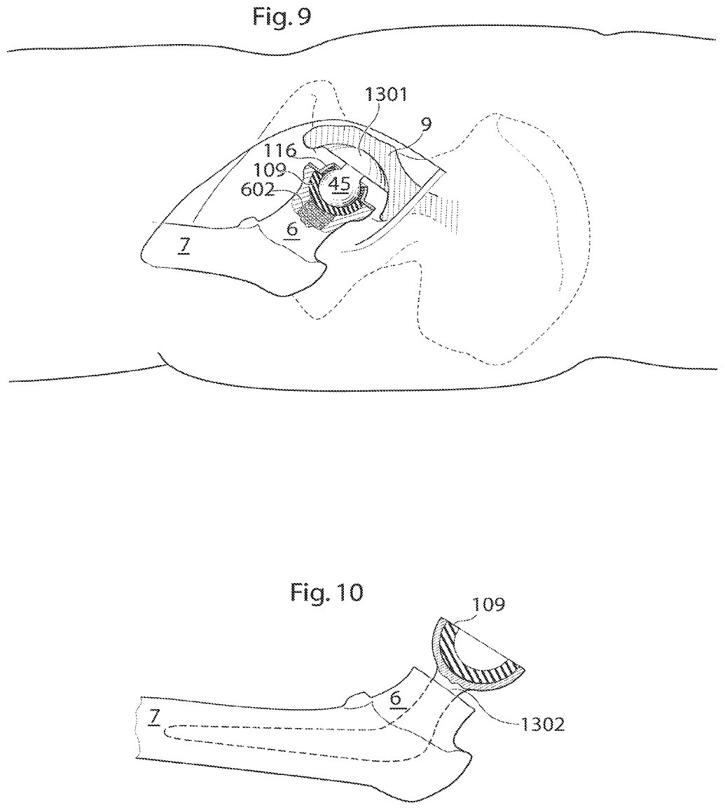

FIG. 9 shows the hip joint in section when the medical device is assembled and in its functional position in the hip joint. The artificial caput femur surface 45 or convex hip joint surface 112 is fixated to the fixation part 1301, which in turn is fixated to the acetabulum 8, The locking element 116 locks the artificial convex caput femur surface 45 in the artificial concave acetabulum surface in the caput 5 and collum femur 6.

FIG. 10 shows an alternative embodiment of concave hip joint surface or concave artificial acetabulum surface of the medical device 109 placed in the femoral bone 7 using a prosthetic stem 1302 adapted to be placed inside of the femoral bone 7, either with bone cement or without.

FIG. 11 shows a frontal view of a human patient when an incision for reaching an area of the hip joint through the pelvic bone in a surgical method has been performed. According to one embodiment the incision 1 is made in the abdominal wall of the human patient. The incision 1 passes through the abdominal wall, preferably rectus abdominis and peritoneum, in to the abdomen of the human patient. In a second embodiment the incision 2 is conducted through the rectus abdominis and in to the pelvic area, below peritoneum. According to a third embodiment the incision 3 is performed just between Illium and the surrounding tissue, an incision 3 which could enable the pelvic bone to be dissected with very little penetration of fascia and muscular tissue. According to a fourth embodiment the incision 4 is made in the inguinal channel. In all of the four embodiments the tissue surrounding the pelvic bone 9 in the area opposite to acetabulum is removed or penetrated which enables the surgeon to reach the pelvic bone 9. It is obvious that the methods described may both be combined or altered reaching the same goal to dissect the pelvic bone on the opposite side of the acetabulum.

FIG. 12 shows a frontal view of a human patient when small incisions for reaching an area of the hip joint through the pelvic bone in a arthroscopic method has been performed. According to a first embodiment the incisions 14 is made in the abdominal wall of the human patient. The small incisions enable the surgeon to insert arthroscopic trocars into the abdomen of the human patient. According to the first embodiment the incisions 14 passes through the abdomen, preferably rectus abdominis and peritoneum, in to the abdomen of the human patient. According to a second embodiment the small incisions 15 is conducted through the rectus abdominis and in to the pelvic area, below peritoneum. According to a third embodiment the small incisions 16 is performed just between Illium and the surrounding tissue, an incision 16 which could enable the pelvic bone to be dissected with very little penetration of fascia and muscular tissue. According to a fourth embodiment the incision 17 is made in the inguinal channel. In all of the four embodiments the tissue surrounding the pelvic bone 9 in the area opposite to acetabulum 8 is removed or penetrated which enables the surgeon to reach the pelvic bone 9.

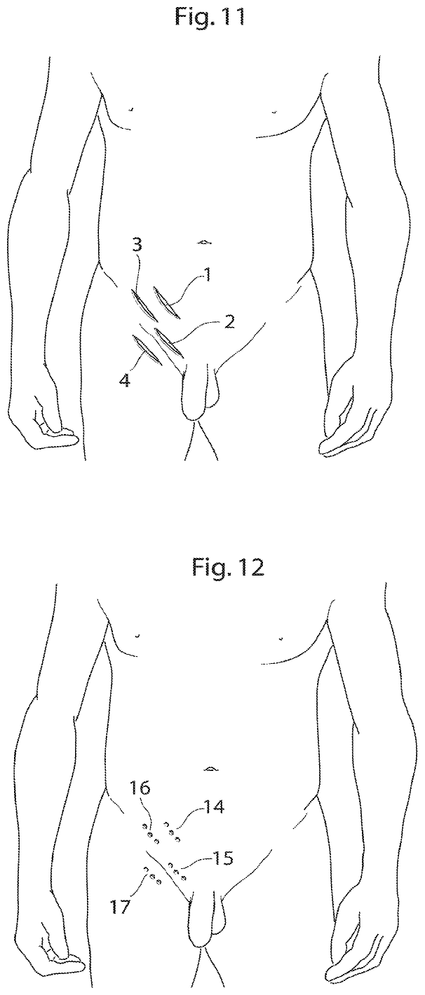

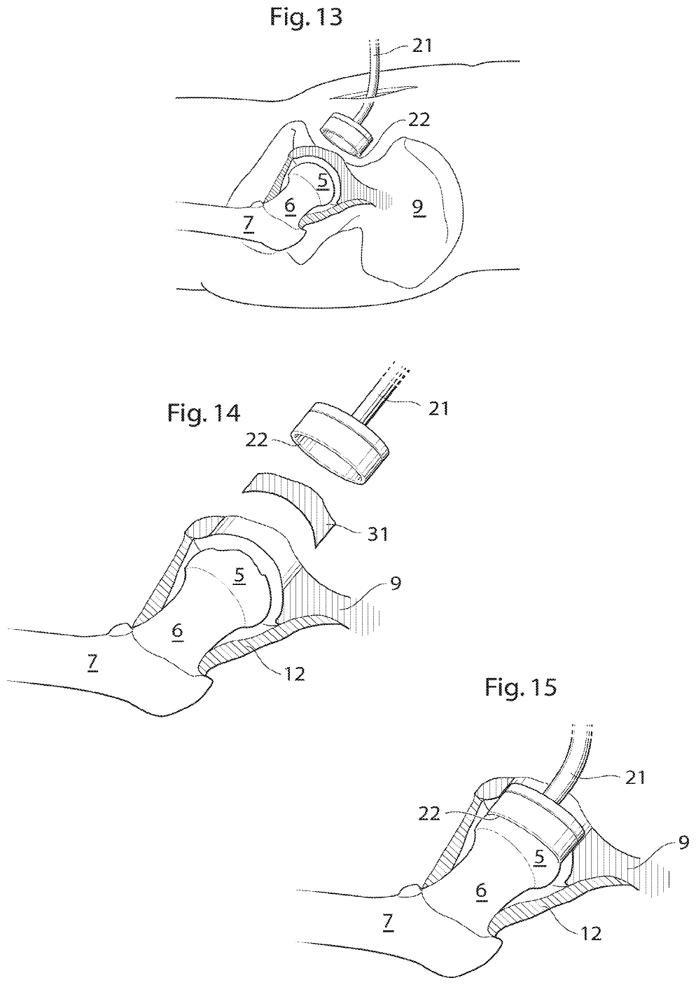

FIG. 13 shows the human patient in section when a medical device for creating a hole 18 in the pelvic bone 9 is inserted through an incision according to any of the embodiments described above. A elongated member 21, which could comprise a part or section adapted to be bent transfers force from an operating device (not shown) to the bone contacting organ 22. The bone contacting organ 22 is placed in contact with the pelvic bone 9 and creates a hole through a drilling, sawing or milling process powered by a rotating, vibrating or oscillating force distributed from the elongated member 21.

FIG. 14 shows the hip joint in section after the medical device for creating a hole 18 in the pelvic bone 9 has created said hole 18. According to this embodiment the hole 18 is created through the removal of a bone plug 31, however it is equally conceivable that said medical device comprises a bone contacting organ 22 adapted to create small pieces of bone, in which case the medical device could further comprise a system for transport of said small pieces of bone.

FIG. 15 shows how the medical device adapted to create a hole is inserted into the hip joint and placed in contact with the caput femur 5. According to this embodiment the medical device for creating a hole in the pelvic bone 9 and the caput femur 5 is the same medical device, however it is equally conceivable that there is a second medical device particularly adapted to create a hole in the caput femur 5.

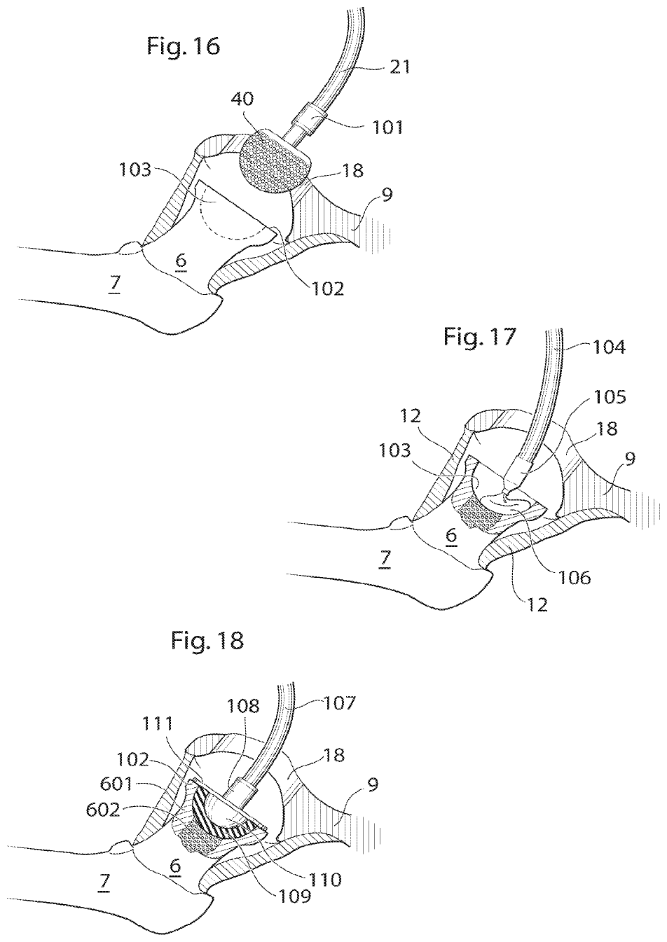

FIG. 16 shows the caput femur 5 after the proximal part has been removed along the section created by the medical device for creating a hole. The removing of the proximal part of the caput femur 5 create a surface of a section 102 in the cortical bone of the caput femur 5. A reamer 40 adapted to create a concave surface 103 in the caput femur 5 is applied to the elongated member 21 through a connecting section 101. According to this embodiment the elongated member 21 is the same as the elongated member used for the medical device adapted to create a hole in the pelvic bone 9, however it is equally conceivable that the elongated member 21 is specifically designed to enable the reaming of the caput femur 5. The reaming in the caput femur and part of the collum femur 6 is mainly performed in the cancellous bone, however that does not exclude the possibility the some of the reaming needs to be performed in the cortical bone of the caput femur 5 and/or the collum femur 6.

FIG. 17 shows the step of applying an adhesive 106 to the concave surface created by the reamer 40. The adhesive 106 is applied by an injecting member 104 comprising an injecting nozzle 105. The adhesive 106 is preferably a biocompatible adhesive such as bone cement. The injecting member 104 is in this embodiment adapted for introduction through a hole 18 in the pelvic bone 9, through the injecting member 104 being bent.

FIG. 18 shows the step of providing a medical device 109 comprising an artificial concave hip joint surface 110. The artificial concave hip joint surface 110 is fixated to the concave surface 103 created in the caput femur 5 and collum femur 6. The medical device 109 comprises a fixation support 111 adapted to anchor said artificial concave hip joint surface 110, to at least one of the caput femur 5 and the collum femur 6. The medical device 109 is adapted to be introduced to the hip joint through a hole 18 in the pelvic bone 9 using a inserting member 107. According to this embodiment the inserting member is bent and thereby adapted to operate through a hole 18 in the pelvic bone 9. The inserting member 107 comprises a connecting member 108 which is adapted to connect to the medical device 109. According to one embodiment the medical device 109 comprises a self lubricating material such as PTFE, however it is also conceivable that said medical device comprises: titanium, stainless steel, Corian, PE, or other acrylic polymers, in which case the medical device could be adapted to be lubricated after insertion in the hip joint.

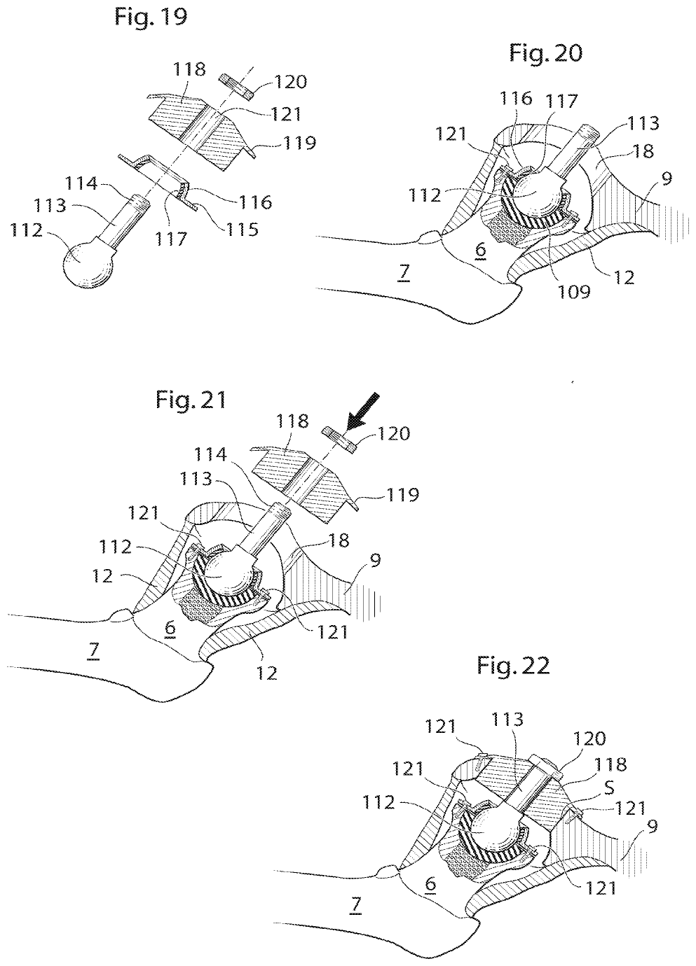

FIG. 19 shows a medical device comprising an artificial convex hip joint surface 112. The artificial convex hip joint surface 112 is adapted to be fixated to the pelvic bone 9, and is adapted to be inserted through a hole 18 in the pelvic bone 9. The medical device comprises a nut 120, comprising threads for securely fixating the medical device to the pelvic bone 9. The medical device further comprises a prosthetic part (which in all embodiments could be a fixating part) 118 adapted to occupy the hole 18 created in the pelvic bone 9 after the medical device has been implanted in the patient. The prosthetic part 118 comprises supporting members 119 adapted to be in contact with the pelvic bone 9 and assist in the carrying of the load placed on the medical device from the weight of the human patient in normal use. Normal use is defined as the same as a person would use a natural hip joint. Further the medical device comprises a locking element 116 comprising a surface 117 adapted to be in contact with the artificial convex hip joint surface 112. The locking element 116 further comprises fixating members 115 which are adapted to assist in the fixation of the locking member 116 to the caput femur 5 or collum femur 6, which in turns fixates the artificial convex hip joint surface 112. The artificial convex hip joint surface 112 is fixated to a attachment rod 113 comprising a thread 114 that corresponds to the thread of the nut 120 in connection with the prosthetic part 118.

FIG. 20 shows the hip joint in section when the artificial convex hip joint surface is fixated in the medical device 109 comprising a concave hip joint surface 110. The convex hip joint surface 112 is secured in place by the locking element 116 which is fixated to the caput femur using screws 121. The surface of the locking element 117 and the concave hip joint surface 117 is placed in connection with the convex hip joint surface and could be made of a friction reducing material such as PTFE or a self lubricating powder material. However it is also conceivable that the connecting surfaces are lubricated using an implantable lubrication system adapted to lubricate the medical device after said medical device has been implanted in the human patient.

FIG. 21 shows the placing of a prosthetic part 118 adapted to occupy the hole 18 created in the pelvic bone 9. The prosthetic part 118 comprises supporting members 119 adapted to be in contact with the pelvic bone 9 and assist in the carrying of the load placed on the medical device from the weight of the human patient. According to the embodiment shown in FIG. 12 the supporting members 119 are located on the abdominal side of the pelvic bone 9, however it is equally conceivable the supporting members 119 are located on the acetabulum side of the pelvic bone 9, in which case they are preferably displaceable for allowing insertion of the prosthetic part 118 through the hole 18 in the pelvic bone 9. Furthermore FIG. 12 shows the fixation of a nut 120 to the attachment rod 113. According to the embodiment shown in FIG. 12 the hole 18 in the pelvic bone 9 is adapted to be larger than the medical device allowing the medical device to be inserted in its full functional size. According to other embodiments the hole 18 is smaller in which case the medical device could comprise of several parts adapted to be connected after insertion in the hip joint, or the medical device could be expandable for insertion through a hole smaller than the full functional size of the medical device. The expandable medical device could be enabled through the elements of the medical device comprising elastic material.

FIG. 22 shows the hip joint in section when all the elements of the medical device has been fixated in the area of the hip joint or its surroundings. The prosthetic part 113 adapted to occupy the hole 18 in the pelvic bone 9 is here fixated with screws 121, however these screws 121 could be assisted or replaced by an adhesive which could be applied to the surface S between the prosthetic part and the pelvic bone 9.

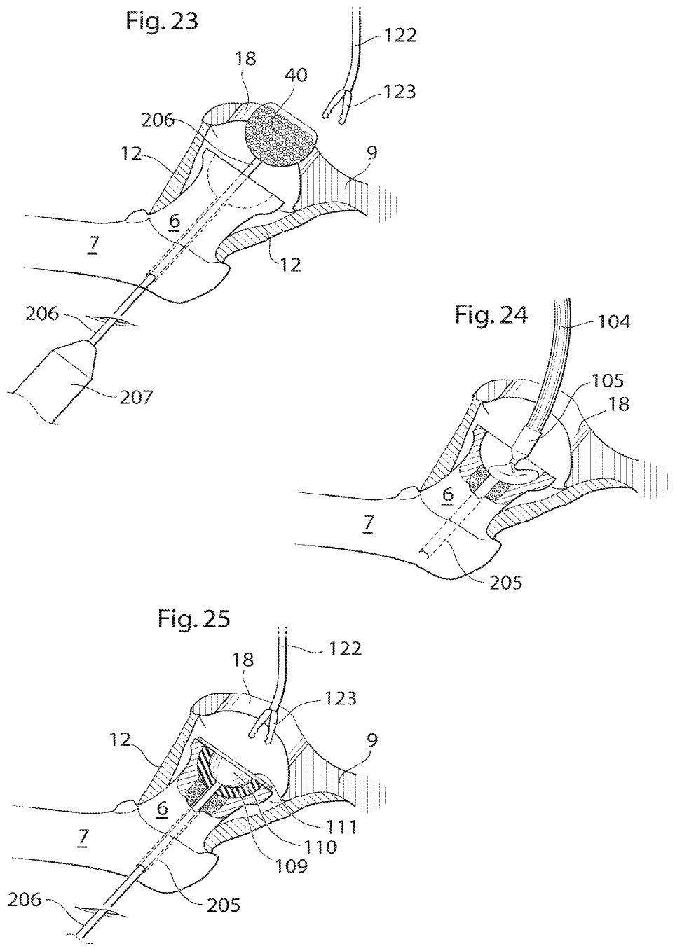

FIG. 23 shows the hip joint in section when the method of supplying the medical device is conducted according to another embodiment. The proximal part of the caput femur has been removed along the section created by the medical device for creating a hole. A reaming member 40 adapted to create a concave surface 103 in the caput femur 5 is here applied to a elongated member 206 which is inserted through a hole 205 going from the lateral side of the thigh, penetrating the cortical bone of the femoral bone 7 propagating along a length axis of the collum femur in the cancellous bone and entering the area of the hip joint. The elongated member 206 is operated using an operating device 207 which could be an electrically powered operating device, a hydraulically powered operating device or a pneumatically powered operating device. The reamer 40 is inserted into the body of the patient through an incision according to FIG. 2 or FIG. 3 and placed in the hip joint through a hole 18 in the pelvic bone 9 using a manipulating device 122 with a gripping member 123 adapted to hold and release said reamer 40. The reaming in the caput femur and part of the collum femur 6 is mainly performed in the cancellous bone, however that does not exclude the possibility the some of the reaming needs to be performed in the cortical bone of the caput femur 5 or the collum femur 6.

FIG. 24 shows the step of applying an adhesive 106 to the concave surface created by the reamer 40. The adhesive 106 is applied by an injecting member 104 comprising an injecting nozzle 105. The adhesive 106 is preferably a biocompatible adhesive such as bone cement. The injecting member 104 is in this embodiment adapted for introduction through a hole 18 in the pelvic bone 9, through the injecting member 104 being bent.

FIG. 25 shows the step of providing a medical device 109 comprising an artificial concave hip joint surface 110. The medical device is according to this embodiment provided with a hole positioned in the length axis of the collum femur 6. The medical device is through the hole adapted to be guided by the elongated member 206 or a guiding rod placed in the hole 205 along a length axis of the collum femur 6. Inserting the medical device into the hip joint while the elongated member 206 or guiding rod runs through the hole of the medical device facilitates the positioning of the medical device and ensures the different parts of the medical device is centered for functioning as a unit. In the embodiment shown in FIG. 16 the medical device 109 is inserted into the hip joint as a single unit, however it is equally conceivable that the medical device 109 is inserted in parts (not shown) which are then connected to form the medical device after implantation in the patient. The artificial concave hip joint surface 110 is fixated to the concave surface 103 created in the caput femur 5 and collum femur 6. The medical device 109 comprises a fixation support 111 adapted to anchor said artificial concave hip joint surface 110, to at least one of the caput femur 5 and the collum femur 6. The medical device 109 is adapted to be introduced to the hip joint through a hole 18 in the pelvic bone 9 using a manipulation device 122 comprising a gripping member 123. According to this embodiment the manipulation device 122 is bent and thereby adapted to operate through a hole 18 in the pelvic bone 9. According to one embodiment the medical device 109 comprises a self lubricating material such as PTFE, however it is also conceivable that said medical device comprises: titanium, stainless steel, Corian, PE, or other acrylic polymers, in which case the medical device could be adapted to be lubricated after insertion in the hip joint.

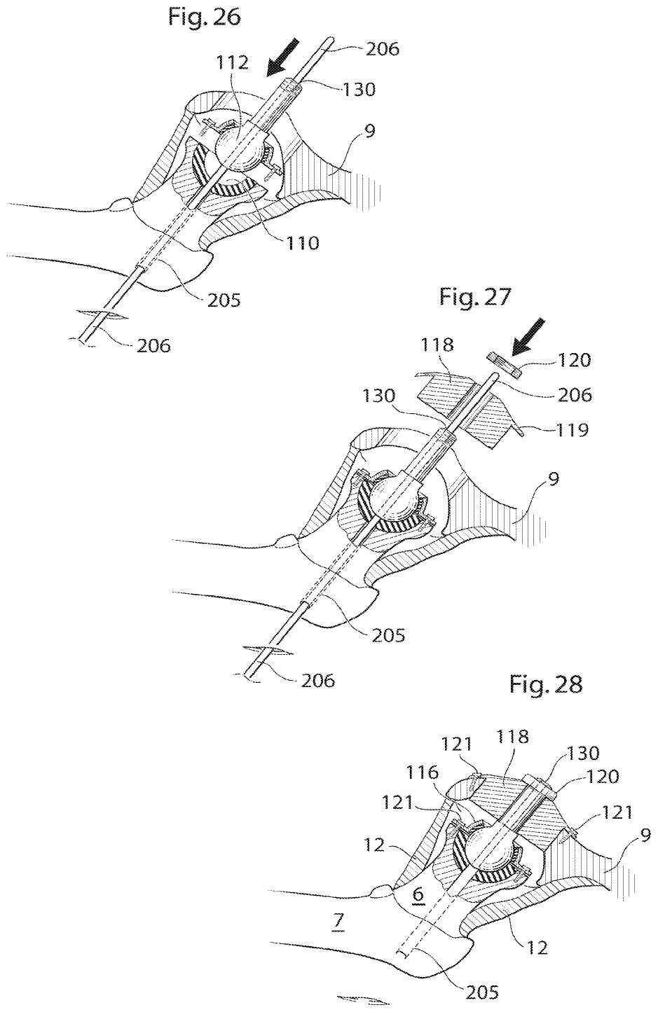

FIG. 26 shows the hip joint in section when the artificial convex hip joint surface is fixated in the medical device 109 comprising a concave hip joint surface 110, the medical device is guided using the elongated member 206 or a guiding rod. The convex hip joint surface 112 is secured in place by the locking element 116 which is fixated to the caput femur using screws 121, the convex hip joint surface is guided using the elongated member 206 or a guiding rod. The surface of the locking element 117 and the concave hip joint surface 110 is placed in connection with the convex hip joint surface and could be made of a friction reducing material such as PTFE or a self lubricating powder material. However it is also conceivable that the connecting surfaces are lubricated using an implantable lubrication system adapted to lubricate the medical device after said medical device has been implanted in the human patient. The elongated member or guiding rod 206 can be adapted to act as a centering rod for centering the at least one artificial hip joint surface inside of the hip joint. According to the embodiment shown the elongated member 206 is inserted through the femoral bone, however according to other embodiments, not shown, the elongated member is positioned inside of the hip joint from the acetabulum side.

FIG. 27 shows the placing of a prosthetic part 118 adapted to occupy the hole 18 created in the pelvic bone 9. The prosthetic part 118 comprises supporting members 119 adapted to be in contact with the pelvic bone 9 and assist in the carrying of the load placed on the medical device from the weight of the human patient. Furthermore FIG. 18 shows the fixation of a nut 120 to the attachment rod 113, which in turn is guided by the elongated member 206 or a guiding rod.

FIG. 28 shows the hip joint in section when all the elements of the medical device has been fixated in the area of the hip joint or its surroundings. The prosthetic part 118 adapted to occupy the hole 18 in the pelvic bone 9 is here fixated with screws 121, however these screws 121 could be assisted or replaced by an adhesive which could be applied to the surface S between the prosthetic part and the pelvic bone 9. The elongated member 206 or guiding rod has been retracted through the incision in the thigh.

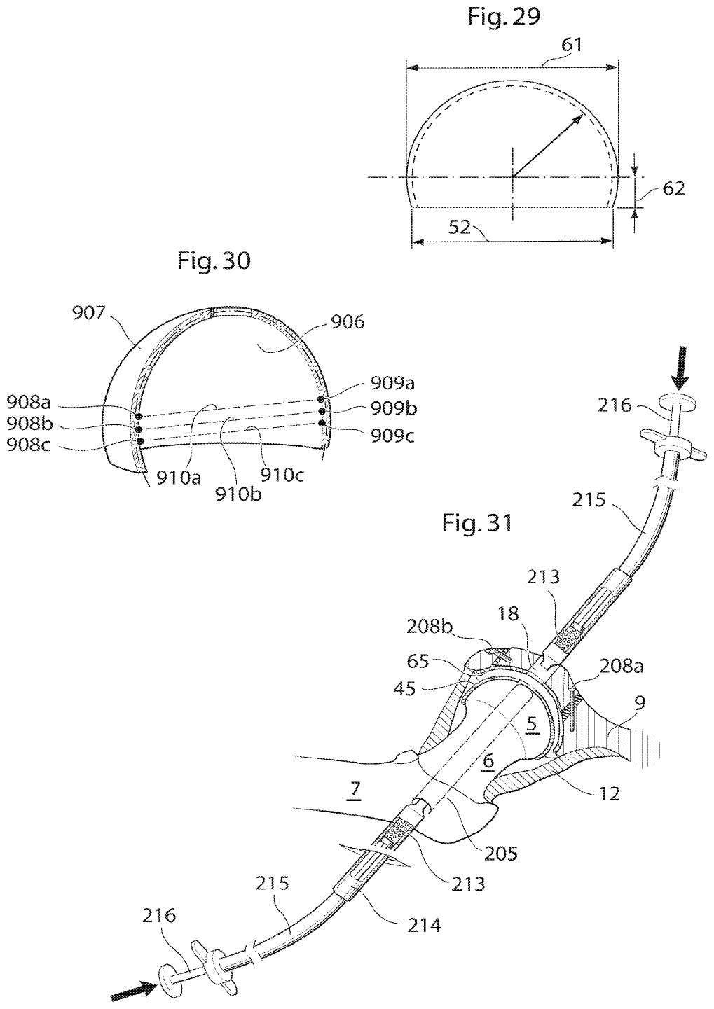

FIG. 29 shows, schematically, how the artificial concave acetabulum surface placed in the collum femur travels beyond the maximum diameter of the convex hip joint surface 112. That is, a cross-sectional distance of the largest opening 52 is smaller than the largest cross sectional distance if an artificial convex caput femur surface, adapted to be placed within said artificial acetabulum surface. According to one embodiment the locking member 116 comprises the part 62 that travels beyond the maximum diameter of the convex hip joint surface, thereby locking the convex hip joint surface in place using the mechanical form of the locking member 116.

FIG. 30 shows the artificial concave acetabulum surface placed in the collum femur according to an embodiment where the artificial hip joint surface comprises an inner surface 906, and an outer surface 907. The inner surface has a first point 908a, a second point 909a, a third point 908b, a fourth point 909b, a fifth point 908c, and a sixth point 909c, all points located on different places along a length axis L of said inner surface 906, wherein: a first straight line 910a, reaching from said first point 908a to said second point 909a is parallel to a second straight line 910b reaching from said third point 908b to said fourth point 909b, which in turn is parallel to a third straight line 910c reaching from said fifth point 908c to said sixth point 909c, wherein: said first and said third straight lines 910a, 910c are of equal length, and wherein said second straight line 910b is longer than said first 910a and said third 910c straight lines and positioned between said first 910a and said third 910c straight lines. The artificial concave acetabulum is thereby passing beyond the maximum diameter of the artificial convex hip joint surface.

The artificial convex caput femur, according to any of the embodiments, could be a solid structure adapted to be inserted into the hip joint as one part, or in multiple parts. The artificial convex solid structure caput femur could comprise at least one of the materials: polyethylene based material, PTFE, Corian, titanium, stainless steel, wolfram, other metal material, a combination of metal material, carbon fiber, boron, a combination of metal and plastic materials, a combination of metal and carbon based material, a combination of carbon and plastic based material, multi-material, wherein one material comprise a flexible material, multi-material, wherein one material comprise an elastic material, multi-material, wherein one material comprising more parts than the other at least one material, PE, and an acrylic polymer. It is also conceivable that the artificial convex solid structure caput femur comprises a self lubricating material. In cases where the artificial convex solid structure caput femur do not comprise a self lubricating material or if the self lubricating material is not sufficient it is conceivable that the medical device is adapted to be lubricated after insertion in the hip joint.

FIG. 31 shows the step of filling the hole 205 in the femoral bone 7 and the hole 18 in the pelvic bone 9. The injecting members 214 is adapted to inject a fluid 213 into said holes 205,18, said fluid 213 could be bone cement or another biocompatible fluid adapted to harden. The injecting members 214 comprises a piston 216 which transfers force through a elongated member 215 which could be flexible for facilitating the surgeon reaching the area where the holes 205,18 is located. After the step of injecting a fluid 213 adapted to harden into the hole 205 is concluded the instruments used in the surgical or arthroscopic method is retracted and the tissue is closed in layers.

According to the above mentioned embodiments the medical device is adapted to be inserted through a hole in the pelvic bone, however it is equally conceivable that the medical device according to any of the embodiment above is adapted to be inserted through a hole in the hip joint capsule or the femoral bone of the human patient.

Please note that any embodiment or part of embodiment as well as any method or part of method could be combined in any way. All examples herein should be seen as part of the general description and therefore possible to combine in any way in general terms.

1.-101. (canceled)

102. A method of treating hip joint osteoarthritis in a human patient by providing at least one hip joint surface, the method comprising the steps of;
fixating an artificial convex caput femur or an artificial convex caput femur surface to the acetabulum or another part of the pelvic bone of the human patient.

103. (canceled)

104. (canceled)

105. The method according to claim 102, the hip joint comprising an acetabulum and a caput femur, the method further comprises the steps of:
cutting the skin of the human patient,
dissecting an area of the pelvic bone on the opposite side from the acetabulum,
creating a hole in said dissected area, said hole passing through the pelvic bone and into the hip joint of the human patient,
inserting said artificial hip joint surfaces into the hip joint through said hole,
closing the hole in the pelvic bone using bone material or a prosthetic part, and
closing, preferable in layers, the hip area of the human patient using sutures or staples.

106. The method according to claim 102, said method further comprises the steps of:
inserting a needle or a tube like instrument into the patient's body,
using the needle or tube like instrument to fill a part of the patient's body with gas and thereby expanding a cavity within said body,
placing at least two arthroscopic trocars in said cavity,
inserting a camera through one of the arthroscopic trocars into said cavity,
inserting at least one dissecting tool through one of said at least two arthroscopic trocars,
dissecting an area of the pelvic bone on the opposite side from the acetabulum,
creating a hole in said dissected area, said hole passing through the pelvic bone and into the hip joint of the human patient, and
providing said artificial hip joint surfaces to the hip joint, through said hole in the pelvic bone of the human patient.

107. The method according to claim 102, wherein said method further comprises the steps of:
a. inserting a centering device into an area of the hip joint,
b. using said centering device to center said at least one hip joint surface in the hip joint.

108. The method according to claim 102, wherein said method further comprises the steps of:
a. inserting an elongated member into an area of the hip joint,
b. manipulating said at least one hip joint surface using said elongated member.

109. The method according to claim 102, wherein said method further comprises the steps of:
a. penetrating the skin of a lateral section of the thigh,
b. creating a hole in the collum femur, along a length axis thereof, reaching an area of the hip joint,
c. placing an elongated member in said hole, wherein said elongated member reaches centrally in said area of the hip joint, and
d. centering said at least one artificial hip joint surface onto said elongated member, wherein said at least one artificial hip joint surface comprises a center hole for guiding said elongated member, and
e. placing said at least one artificial hip joint surface in a functional position in the hip joint.

110. The method according to claim 102, wherein said method further comprises the steps of:
a. penetrating the skin of the thigh of the human patient,
b. penetrating the hip joint capsule,
c. providing at least one hip joint surface,
d. closing the hip joint capsule, and
e. suturing or stapling the skin of the human patient

111. A method of treating hip joint osteoarthritis in a human patient by providing at least one hip joint surface, the method comprising the steps of;
fixating an artificial concave acetabulum or an artificial concave acetabulum surface to at least one of; the caput femur, the collum femur and the femoral bone of the human patient.

112. The method according to claim 111, the hip joint comprising an acetabulum and a caput femur, the method further comprises the steps of:
cutting the skin of the human patient,
dissecting an area of the pelvic bone on the opposite side from the acetabulum,
creating a hole in said dissected area, said hole passing through the pelvic bone and into the hip joint of the human patient,
inserting said artificial hip joint surfaces into the hip joint through said hole,
closing the hole in the pelvic bone using bone material or a prosthetic part, and
closing, preferable in layers, the hip area of the human patient using sutures or staples.

113. The method according to claim 111, said method further comprises the steps of:
inserting a needle or a tube like instrument into the patient's body,
using the needle or tube like instrument to fill a part of the patient's body with gas and thereby expanding a cavity within said body,
placing at least two arthroscopic trocars in said cavity,
inserting a camera through one of the arthroscopic trocars into said cavity,
inserting at least one dissecting tool through one of said at least two arthroscopic trocars,
dissecting an area of the pelvic bone on the opposite side from the acetabulum,
creating a hole in said dissected area, said hole passing through the pelvic bone and into the hip joint of the human patient, and
providing said artificial hip joint surfaces to the hip joint, through said hole in the pelvic bone of the human patient.

114. The method according to claim 111, wherein said method further comprises the steps of:
a. inserting a centering device into an area of the hip joint,
b. using said centering device to center said at least one hip joint surface in the hip joint.

115. The method according to claim 111, wherein said method further comprises the steps of:
a. inserting an elongated member into an area of the hip joint,
b. manipulating said at least one hip joint surface using said elongated member.

116. The method according to claim 111, wherein said method further comprises the steps of:
a. penetrating the skin of a lateral section of the thigh,
b. creating a hole in the collum femur, along a length axis thereof, reaching an area of the hip joint,
c. placing an elongated member in said hole, wherein said elongated member reaches centrally in said area of the hip joint, and
d. centering said at least one artificial hip joint surface onto said elongated member, wherein said at least one artificial hip joint surface comprises a center hole for guiding said elongated member, and
e. placing said at least one artificial hip joint surface in a functional position in the hip joint.

117. The method according to claim 111, wherein said method further comprises the steps of:
a. penetrating the skin of the thigh of the human patient,
b. penetrating the hip joint capsule,
c. providing at least one hip joint surface,
d. closing the hip joint capsule, and
e. suturing or stapling the skin of the human patient

118. A method of treating hip joint osteoarthritis in a human patient by providing at least two hip joint surfaces, the method comprises the steps of;
fixating an artificial convex caput femur or an artificial convex caput femur surface to the acetabulum or another part of the pelvic bone of the human patient, and
fixating an artificial concave acetabulum or an artificial concave acetabulum surface to at least one of; the caput femur, the collum femur and the femoral bone of the human patient.

119. The method according to claim 118, the hip joint comprising an acetabulum and a caput femur, the method further comprises the steps of:
cutting the skin of the human patient,
dissecting an area of the pelvic bone on the opposite side from the acetabulum,
creating a hole in said dissected area, said hole passing through the pelvic bone and into the hip joint of the human patient,
inserting said artificial hip joint surfaces into the hip joint through said hole,
closing the hole in the pelvic bone using bone material or a prosthetic part, and
closing, preferable in layers, the hip area of the human patient using sutures or staples.

120. The method according to claim 118, said method further comprises the steps of:
inserting a needle or a tube like instrument into the patient's body,
using the needle or tube like instrument to fill a part of the patient's body with gas and thereby expanding a cavity within said body,
placing at least two arthroscopic trocars in said cavity,
inserting a camera through one of the arthroscopic trocars into said cavity,
inserting at least one dissecting tool through one of said at least two arthroscopic trocars,
dissecting an area of the pelvic bone on the opposite side from the acetabulum,
creating a hole in said dissected area, said hole passing through the pelvic bone and into the hip joint of the human patient, and
providing said artificial hip joint surfaces to the hip joint, through said hole in the pelvic bone of the human patient.

121. The method according to claim 118, wherein said method further comprises the steps of:
a. inserting a centering device into an area of the hip joint,
b. using said centering device to center said at least one hip joint surface in the hip joint.
